(19) United States
(12) Patent Application Publication
(10) Pub. No.: US 2005/0049515 A1
(43) Pub. Date: Mar. 3, 2005

(54) Electrode belt for acquisition, processing and transmission of cardiac (ECG) signals

(76) Inventors: Dale Julian Misczynski, Austin, TX (US); Vladislav Bukhman, East Northport, NY (US); Sergii Tymoshok, Kiev (UA); Dmytro Tymoshok, Kiev (UA); Oleg Sychov, Kiev (UA)

Correspondence Address: Dale J. Misczynski, 1800 Barton Creek Blvd., Austin, TX, 78735-1606, US

(21) Appl. No.: 10/901,588

(22) Filed: Jul. 29, 2004

Related U.S. Application Data
(60) Provisional application No. 60/491,409, filed on Jul. 31, 2003.

(51) Int. Cl. A61B 5/0402

(52) U.S. Cl. 600/509

(57) ABSTRACT

The present invention relates to an electrode chest belt for acquisition, processing and transmission of cardiac (ECG) signals. The chest belt comprises a flexible belt, which adjusts to accommodate individuals of varying sizes, and two or more electrodes that are inserted in soft flexible pads, placed in predetermined positions on the belt, and connected to an embedded electronic processing unit wherein the acquired cardiac signals are amplified, filtered, digitized and transmitted to any number of other devices wherein additional processing and storage of the signals may take place.

### CROSS REFERENCE TO RELATED APPLICATIONS

This patent application is based upon Provisional Patent Application Ser. No. 60/491,409 filed on Jul. 31, 2003

### BACKGROUND

Medical professionals routinely seek to monitor the electrical currents produced by a patient's heart. An electrocardiogram (ECG) is a recording of these currents. The normal ECG is composed of a P wave, a “QRS complex,” and a T wave. An abnormality in the ECG, such as abnormal rhythms, ST deviation or Long “QT” interval, may be indicative of a medical problem. Consequently, ECG's are valuable tools in monitoring an individual's health.

ECG's are acquired with electrodes and leads. “Lead I” is an example of a lead wherein an electrode is connected to the patient's right arm and another electrode is connected to the left arm. A Lead I ECG represents the changes in the electric potential, over time, between these two electrodes. Traditionally, six electrodes are placed at various locations on the body to generate three standard leads (I to III) and three augmented leads (aVR, aVL and aVF). Six more electrodes are placed at designated positions on the chest to produce pre-cordial chest leads (V1-V6). Together these leads make up a traditional 12-lead ECG. The different placement of electrodes in different leads produces different views (angles) of the heart's electrical activity. Many lead configurations are possible thereby making many different views of this activity possible. Modified Central Lead 1 (MCL1) as well as posterior leads V7, V8 and V9 are examples of alternative configurations to those found in a standard 12-lead ECG.

Considering the multitude of options for leads and electrode placement, a medical professional must evaluate how many of the leads are necessary for any given task. For example, a detailed diagnostic examination of a patient might require all twelve traditional leads. However, prolonged monitoring of the heart might be accomplished using only Lead I to provide a “big picture” of the heart's electrical activity. Longer-term monitoring activities might be limited to Lead I because only a few parameters, such as a change in rhythm or prolongation in a certain interval, will provide all the necessary information. Opting to use fewer leads forfeits some detail but, at the same time, gains some convenience, because while fewer views of the heart's electrical activity are obtained, fewer electrodes need to be applied to the patient.

This is important because applying electrodes to the patient can be quite a task. The electrodes must be placed in the proper location on the patient. Location is very important because different positioning of electrodes affects appearances of the ECG waves. An improper positioning of the electrodes will not produce a signal that the examiner can compare to known benchmarks. Improperly positioned electrodes may also result in electromagnetic noise, signal artifacts and disruption of electrical conductivity: all of which can result in the misinterpretation of ECG data. Some factors that help determine electrode location include what the specific topic of study is. For example, electrodes placed on the chest produce signals that are more indicative of ischaemic changes in the heart. Interference due to electrical activity from muscles, other than the heart tissue, is also a consideration for electrode placement. Still, while proper electrode positioning is important for all studies, proper positioning is more critical for a detailed diagnostic study than for a more long-term monitoring activity.

Applying electrodes to the patient is a difficult task because, in addition to the aforementioned location concerns, the electrodes need to be in good contact with the skin in order to acquire a good signal. Consequently, the skin may need to be prepared by shaving or chaffing. Also, the electrodes traditionally need to be connected to a plethora of wires that are in turn connected to monitoring equipment, such as an external ECG acquisition and processing device, which needs to be operated by medical personnel. Finally, in prolonged monitoring exercises, this “set-up” must be able to last over extended periods of time because the examiner is most interested in analyzing how the different ECG's change over time in relation to one another. In the end, obtaining the signals is inconvenient because it is a time-consuming practice that requires the know-how of a skilled medical professional.

The inconvenience is also made worse by the wires, electrodes and other gear which act to constrain the patient. This makes the all important changes that occur over longer periods of time, like those which are the focus of monitoring exams, more difficult to obtain because the cumbersome equipment does not allow the patient to carry on with a normal routine.

The medical community has yet to overcome these problems of convenience and complication. Consequently, equipment that can acquire ECG's over a prolonged period of time, without being overly cumbersome or requiring expert preparation, is desired. Addressing one part of the problem, there are a number of methods for the pre-positioned placement of pre-cordial leads: U.S. Pat. Nos. 4,121,575, 4,233,987, 4,328,814, 5,042,481, 5,168,875, 5,184,620 and 6,205,346. Unfortunately, these methods require electrode application by trained professionals. Also, the electrodes must still be connected to wires and an external ECG device, thus constraining the patient from freedom of movement.

The present invention overcomes the prior art's problematic need for cumbersome equipment and professional assistance by providing a new approach to the placement of electrodes and the acquisition, processing and transmission of electrical heart activity signals for the monitoring of ECG's.

### BRIEF DESCRIPTION OF THE DRAWINGS

A better understanding of the present invention can be obtained when the following detailed description is considered in conjunction with the following drawings, in which:

FIG. 1 is an illustration of an electrode belt used for the acquisition, processing and transmission of ECG signals;

FIG. 2 is a frontal view of the electrode belt with a signal processing unit and electrodes with soft flexible pads; and

FIGS. 3-6 are schematic diagrams of the signal processing unit.

### DETAILED DESCRIPTION OF THE DRAWINGS

In the following description, numerous specific details are set forth to provide a thorough understanding of the present invention. However, it will be obvious to those skilled in the art that the present invention may be practiced without such specific details. For the most part, details concerning specific non-essential materials and the like have been omitted inasmuch as such details are not necessary to obtain a complete understanding of the present invention and are within the skills of persons of ordinary skill in the relevant art.

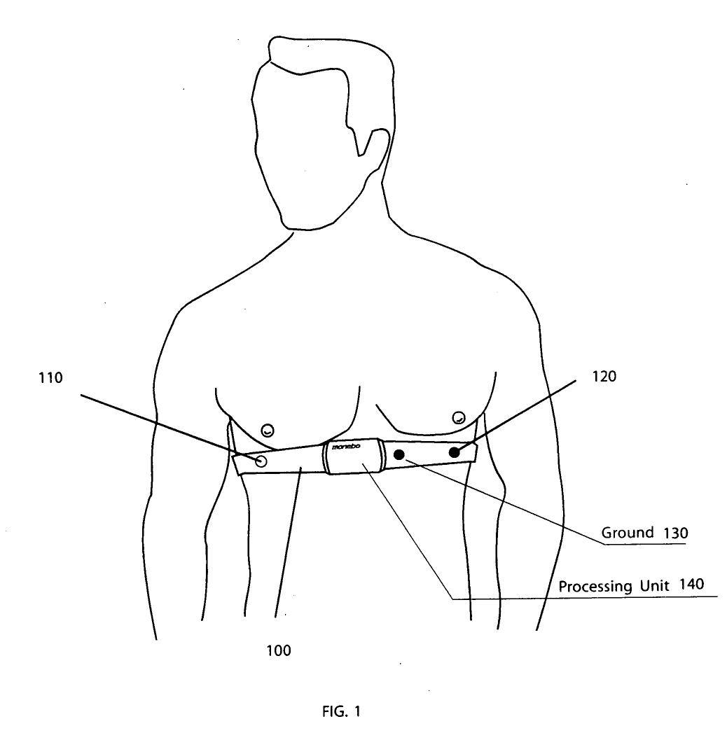

An embodiment of the invention is shown in FIG. 1 wherein a belt 100, with electrodes 110, 120, 130 and signal processing unit (“SPU”) 140 are shown. Cardiac signals are acquired through the electrodes 110, 120, 130 and are then conveyed over wiring embedded in the belt 100 to the SPU 140 where processing and transmission of the ECG's takes place.

The belt 100 is made, for example, from a flexible material that is light, soft, porous, non-slipping and comfortable to wear. The belt may be tubular providing a conduit for the wires that connect the electrodes 110,120, and 130 to the SPU 140. In another embodiment, the belt 100 may be constructed from several layers that encompass the wires that connect the electrodes 110,120, and 130 to the SPU 140. The wires may interface the SPU 140 using standard DB9 (male and female) Belkin connectors. The SPU 140 is inserted in a pocket that can be opened to allow the SPU 140, after being disconnected from the wires that interface the electrodes 110,120, and 130, to be removed from the belt 100. This allows for disposal of the belt 100 after a single use or when a different patient will be monitored. The SPU 140 can then be easily connected to a new belt 100 to allow reuse of the SPU 140.

Three electrodes 110, 120, 130 are shown. One serves as a neutral electrode 130 while the other two electrodes 110, 120 are located at the modified Standard Lead I position. Lead I is modified in such a manner that all three electrodes 110, 120, 130 are placed substantially in-line on the belt. A more traditional Lead I would place three electrodes in more of a triangular pattern. For example, an electrode would be placed on each shoulder with the third electrode on the patient's leg. While the signal generated from the in-line configuration is not exactly like one generated from a traditional Lead I configuration, the two signals are similar to one another. The in-line configuration still produces a quality signal which satisfies the “big picture” requirements of prolonged monitoring examinations such as, for example, change in rhythm or changes in certain intervals such as the “QT” interval. With the belt 100, the left “viewing” electrode 120 is positioned in close proximity to the heart's left ventricle, thus providing signals upon which transient arrhythmia events are more easily detected. This position is also good for monitoring ischaemic changes in this area. Thus, the in-line configuration produces quality signals while also being conducive to placement on a single belt 100.

A major advantage of the invention is that a layman can easily put the belt 100 on. The belt 100 can be placed almost anywhere on the torso so long as the electrodes 110, 120 are approximately equidistant from the sternum. The flexibility in positioning of the belt 100 is possible because the monitoring study goals may be accomplished with one “big picture” signal of good quality. This signal may be derived from a number of positions on the torso. Thus, the configuration of the electrodes 110, 120, 130 on the belt 100 provides for proper electrode placement. As discussed above, in the prior art, such proper placement usually requires the assistance of skilled medical personnel. Furthermore, the tension in the belt holds the electrodes 110, 120, 130 in good contact with the skin thereby limiting the need for skin preparation, whether it be by chaffing the skin or otherwise. Also, the tension in the belt 100 keeps the belt 100 in the same position on the torso throughout the monitoring session. Consequently, the belt 100 negates the need for application of the device by skilled medical personnel.

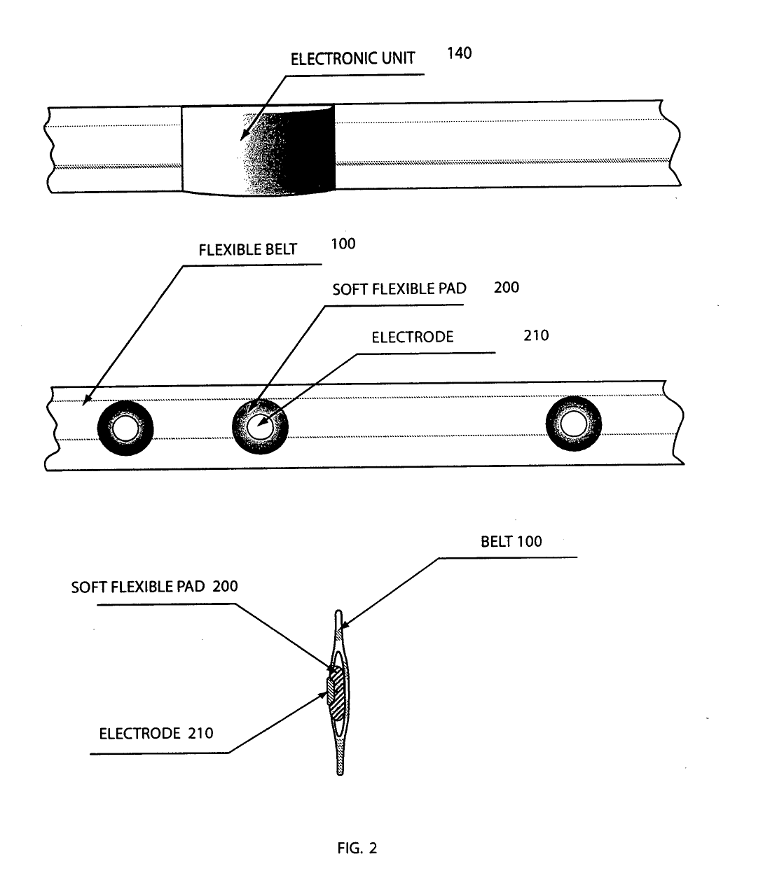

Now referring to FIGS. 2A and 2B, the electrodes 110, 120, 130 are inserted into soft, flexible pads 200 constructed from, for example, silicon rubber that is commonly used for medical applications. (Suitable rubber is manufactured by Vesta, Inc., 5400 West Franklin Drive, Franklin, Wis. 53132). The electrode surface 210 is still in direct contact with the skin while the pad 200 encircles the electrode 210. This helps reduce artifacts associated with muscle contraction because the electrode 210 stays in contact with the skin while the pad 200 deforms in correspondence with body and belt 100 movement. The improved skin contact further negates the need for skilled assistance in skin preparation and application of the electrodes 110, 120, 130.

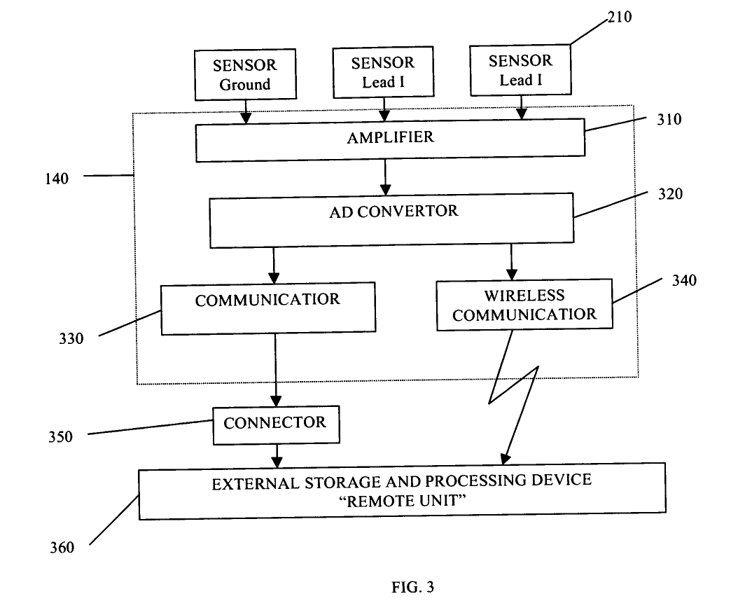

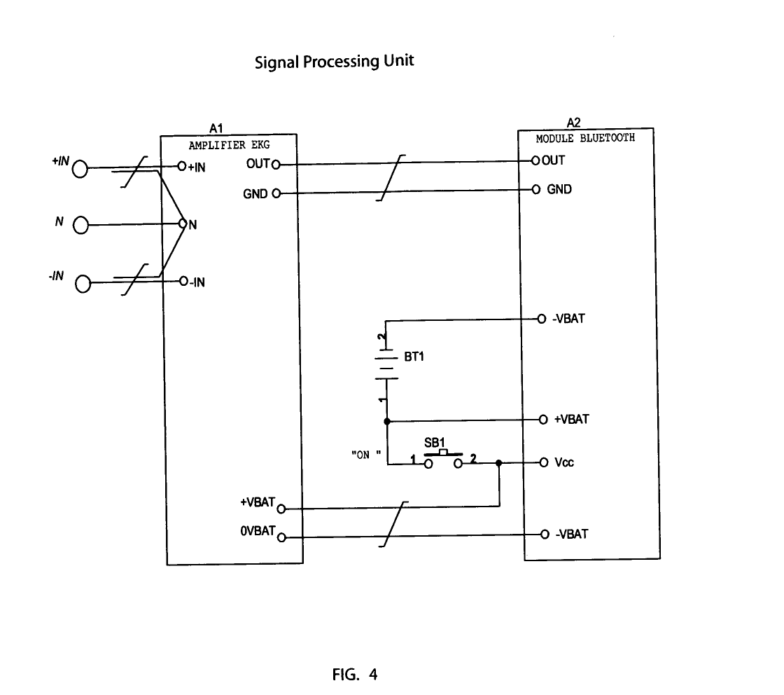

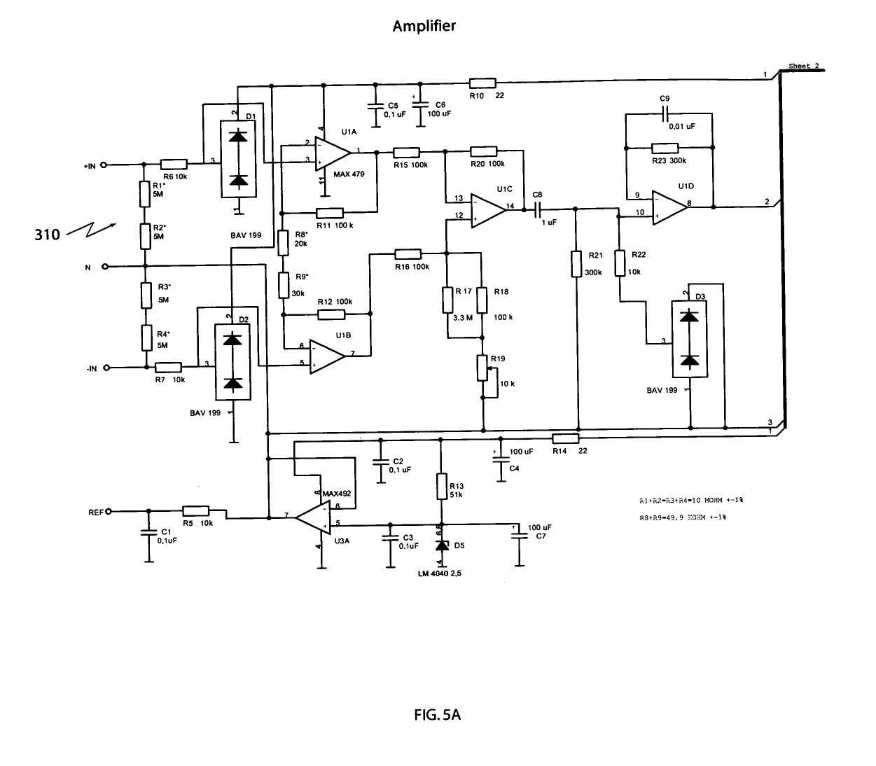

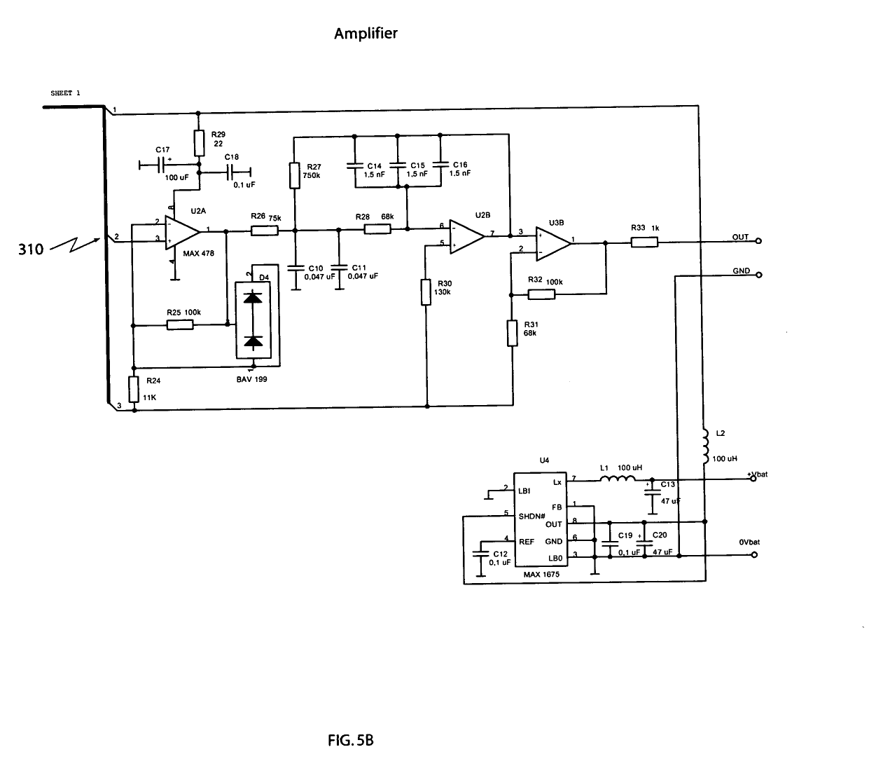

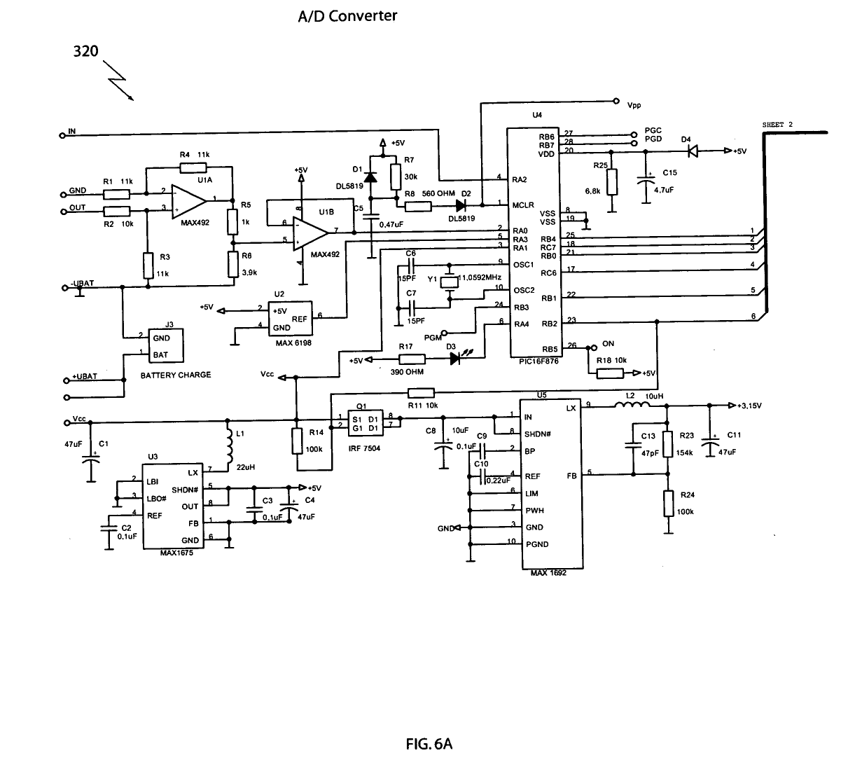

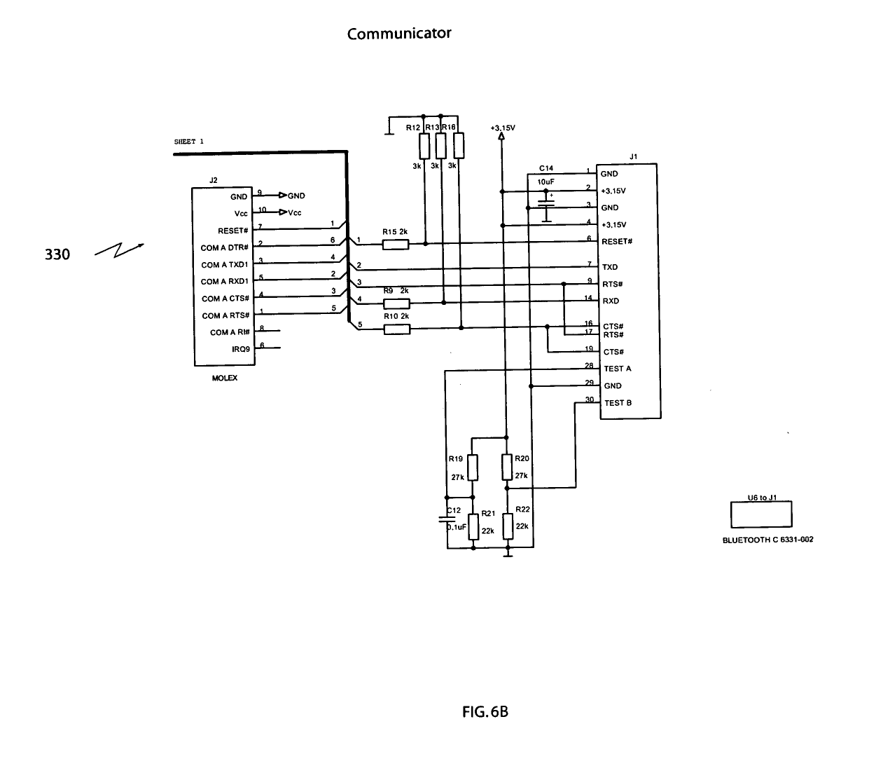

As shown in FIG. 3 and FIG. 4, the SPU 140 comprises an amplifier 310 (see FIGS. 5A and 5B), where amplification and filtering occurs, analog-to-digital (A/D) converter 320 (see FIG. 6A) and a communicator 330 (see FIG. 6B). The purpose of the amplification stage is to add gain into the signal path as well as a moderate degree of band pass filtering. The frequency response should be approximately 0.5 to 1000 Hz. The second stage of amplification is to electrically combine the two signals into a composite signal. Common operational amplifiers and differential amplifiers are used for this purpose.

The analog-to-digital converter is also a common device, typically based upon a eight bit microprocessor. It should be able to sample at preset rates from 100 to 1000 samples per second.

As shown in FIG. 3, the SPU 140 is embedded in the belt 100 and can send and receive digitized data to or from any remote ECG storage and/or processing devices (“remote unit”) 360. This transmission may be through wire 330, 350 or wireless 340 means. The transmission standard is not a limitation in that, for example, Bluetooth or Wi-Fi are both viable options. Raw digitized ECG data can also be stored in the SPU 140 and downloaded for further processing at a later time.

The remote unit 360 may be a unit that is designed and dedicated to ECG processing or it may be a more generic device such as a PC, PDA or smart phone that is equipped with ECG acquisition and analysis software with which the cardiac data can be processed.

The SPU 140 is kept physically small by reserving the majority of the signal processing for the remote unit 360. By doing so, the SPU's 140 circuitry does not require a great deal of power. This allows a relatively small power supply to power the SPU, thereby providing a lower weight device that is not cumbersome to the patient.

The invention provides mobility and freedom-of-movement advantages because the electrodes 110, 120, 130, the wiring from the electrodes 110, 120, 130 to the SPU 140, and the SPU 140 itself, are located within the belt. The small, lightweight SPU 140 provides signal processing and storage capabilities that are normally located on large, external monitoring equipment. Consequently, the individual wearing the invention can participate in an active lifestyle while still obtaining high quality ECG signals that can be stored and analyzed without the need for cumbersome equipment that requires the assistance of skilled medical personnel to apply and use. Finally, the soft flexible pads 200, coupled with the adjustable belt 100 that can be tightened to ensure a snug yet comfortable fit, enable the acquisition of good signals by ensuring good electrode/skin contact even when the individual is very mobile and active. This provides an ideal solution to longer term monitoring exams where the patient may leave the laboratory setting.

Other embodiments of the invention are not limited to cardiac signals or to human subjects. The device is, for example, designed to acquire, process and transmit signals from a mobile subject with minimal assistance from a skilled medical professional. Thus, acquisition, processing and transmission of EMG signals from a dog or cat are but one example of an alternative application of the invention.

Another embodiment allows for greater use of Wi-Fi environments. For example, as cities continue to increasingly provide “hot spots” for wireless communication devices to be able to transmit and receive wireless signals, the invention will allow a subject to remain “monitored” as she walks throughout the “hot spot.” In other words, the invention continually acquires, processes and transmits cardiac data to a remote unit that receives the transmitted signal and, upon further analysis, could alert medical personnel to adverse changes in the patient's condition.

Another embodiment uses only two electrodes whereby a unipolar signal is monitored. Other embodiments may use more electrodes in various electrode configurations to provide varying levels of detail and perspectives regarding the electrical activity of the heart.

Although the present invention and its advantages have been described in detail, it should be understood that various changes, substitutions and alterations can be made herein without departing from the spirit and scope of the invention.

1. Apparatus for monitoring physiologic signals comprising:
a belt;
electrodes, connected to said belt, for acquiring physiologic signals wherein said electrodes are substantially in line with each other;
means for signal processing connected to said belt; and
wires connecting said electrodes to said means for signal processing.

2. The apparatus of claim 1, wherein said means for signal processing comprises:
means for amplification of signals;
means for filtering of signals;
means for digitization of signals;
memory for storage of signals; and
means for transmitting signals to a remote unit wherein transmitted signals are analyzed.

3. The apparatus of claim 2 wherein said transmission of signals occurs within 1 second of said acquisition of said signals.

4. The apparatus of claim 3 wherein said transmission of signals is wireless.

5. The apparatus of claim 2 wherein said transmission of signals occurs within 5 seconds of said acquisition of said signals.

6. The apparatus of claim 5 wherein said transmission of signals is wireless.

7. The apparatus of claim 1 weighing less than one pound.

8. The apparatus of claim 1 weighing less than two pounds.

9. The apparatus of claim 8 wherein said transmission of signals occurs within 5 seconds of said acquisition of said signals.

10. The apparatus of claim 1 weighing less than four pounds.

11. The apparatus of claim 1 wherein said electrodes reside substantially within said belt.

12. The apparatus of claim 1 wherein said electrodes reside substantially on flexible disks.

13. The apparatus of claim 2 wherein said remote unit is a PDA, smartphone or laptop computer.
